(19) United States
(12) Patent Application Publication
(10) Pub. No.: US 2013/0309333 A1
(43) Pub. Date: Nov. 21, 2013

(54) METHOD AND COMPOSITION FOR TREATING FELINE SKIN DISORDERS

(71) Applicant: Linda Brooks, Houston, TX (US)

(72) Inventor: Linda Brooks, Houston, TX (US)

(21) Appl. No.: 13/673,949

(22) Filed: Nov. 9, 2012

Related U.S. Application Data
(60) Provisional application No. 61/557,473, filed on Nov. 9, 2011.

(51) Int. Cl. A61K 36/889

(52) U.S. Cl. CPC A61K 36/889
USPC 424/727

(57) ABSTRACT

In a new method of treating feline skin disorders, an area of abnormal skin is identified on a cat, and coconut oil is applied to the area of abnormal skin. To apply the coconut oil, it is applied to a user's fingertips, the user's fingers are pushed through the cat's fur until they contact the cat's skin, and then the user's fingertips are brushed against the skin of the cat against the lie of the fur. The user may continue the application until the coconut oil has been applied to the full area of abnormal skin. Application may be repeated once daily until symptoms of skin disorder completely abate. The coconut oil may be pure or mixed with one or more additional ingredients for aroma, appearance, or texture. The amount of coconut oil applied may be less than ¼ of a teaspoon.

This application claims priority to U.S. provisional patent application No. 61/557,473, filed Nov. 9, 2011, which is hereby incorporated herein in its entirety.

### BACKGROUND

The present invention relates generally to veterinary medicine and specifically to feline dermatology.

Numerous existing treatments for feline skin disorders are sold exclusively through veterinarians or through a variety of chain and boutique pet stores. However, these treatments are frequently ineffective, expensive, rejected by cats, and/or have offensive smells or feels. Needs exist for improved methods for treating feline skin disorders.

### SUMMARY

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In certain embodiments, the disclosed embodiments may include one or more of the features described herein.

In a new method of treating feline skin disorders, coconut oil is applied to the affected skin of a cat with a user's fingertips. Rapid improvement is unexpectedly achieved for a wide variety of feline skin ailments, with full relief of symptoms.

In a new method of treating feline skin disorders, coconut oil is applied to the skin of a cat. An area of abnormal skin may first be identified on the cat, and the coconut oil then applied to the area of abnormal skin. The cat's fur may then be smoothed down in the direction of growth. The coconut oil may be applied by applying coconut oil to a user's fingertips, pushing the user's fingers through the cat's fur until they contact the cat's skin, and then brushing the user's fingertips against the skin of the cat against the lie of the fur. The user may continue the application until the coconut oil has been applied to the full area of abnormal skin. The coconut oil may be pure, or may be mixed with one or more additional ingredients for aroma, appearance, or texture, such as herbs (for example lavender, rosemary or thyme), food coloring, glycerine, or almond extract. The coconut oil may be 50% or more by weight of the mixture. The amount of coconut oil applied may be less than ¼ of a teaspoon.

The application of coconut oil to the user's fingertips may be accomplished by applying coconut oil to the fingertips of one of the user's hands and then rubbing those fingertips against the fingertips on the user's other hand, applying coconut oil to the fingertips of one of the user's hands and then rubbing the thumb of that hand against the other fingertips, or running the tips of the fingers of one hand across the surface of the coconut oil until the fingertips are coated.

The coconut oil may be melted before application to the cat, for example using body heat and/or friction. The coconut oil may be applied to the middle three fingers of the user's hand. Three strokes against the cat's skin with the fingers of both the user's hands may be sufficient to accomplish a full application. Application may be repeated once daily until symptoms of skin disorder completely abate, and then whenever symptoms return, or every 4-5 days.

A new feline skin disorder treatment composition includes coconut oil and one or more additional ingredients for aroma, appearance, or texture, with the coconut oil mixed with the one or more additional ingredients such that the coconut oil is 50% or more of the mixture by weight. The composition may include instructions for applying the composition to a cat's skin to treat a feline skin disorder.

These and other objectives and features of the invention are apparent in the disclosure, which includes the above and ongoing written specification.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 is a flowchart illustrating a feline skin disorder treatment method, in an embodiment.

### DETAILED DESCRIPTION

A new method for treating feline skin disorders will now be disclosed in terms of various exemplary embodiments. This specification discloses one or more embodiments that incorporate features of the invention. The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. When a particular feature, structure, or characteristic is described in connection with an embodiment, persons skilled in the art may effect such feature, structure, or characteristic in connection with other embodiments whether or not explicitly described.

The embodiments described, and their detailed construction and elements, are merely provided to assist in a comprehensive understanding of the invention. Thus, it is apparent that the present invention can be carried out in a variety of ways, and does not require any of the specific features described herein. Also, well-known functions or constructions are not described in detail since they would obscure the invention with unnecessary detail.

In a new method for treating feline skin disorders, a user takes coconut oil, which is solid at room temperature, and rubs it on the user's fingertips until the coconut oil has partially or completely melted and the fingertips are coated. For a fast and effective treatment, the fingertips of the middle three fingers of one hand may be coated and rubbed upon the corresponding fingers of the other hand. If the user desires, only a single hand may be used for application, with the thumb used to help rub and coat the fingertips with the product. Only a small amount of coconut oil may be necessary for performing the treatment, as less than ¼ of a teaspoon is often sufficient for treatment of a full-size house cat (generally 9-11 lbs). The user may merely run the tip of the fingers of one hand across and back and forth upon the surface of the oil, which readily melts and coats the fingertips.

The fingertips may then be pushed through a cat's fur until they contact the cat's skin, and the coated fingertips may then be brushed against the cat's skin. The coconut oil is applied topically to the skin via the process of touching and stroking. This application process is repeated (using more product if necessary) until the coconut oil has been applied to the full area of the affected skin. Stroking against the lie (direction of growth) of the cat's fur is typically most effective, resulting in more of the coconut oil applied to the cat's skin and less to the fur. After application, the cat's fur may be smoothed down in the direction of growth to leave a smooth coat. This whole process can be completed very quickly, with a typical application requiring only about three strokes with both hands. Thus, there is little risk of the cat becoming agitated and attempting to leave before the treatment is complete.

This application causes an immediate improvement in the appearance of the cat, making the fur softer and more glossy. The application may be repeated daily until symptoms of the skin disorder completely abate. It has been found that, for some disorder, as few as four initial daily applications may be required for complete cessation of symptoms to occur. For some disorders, symptoms may begin again 4-5 days after an application, necessitating a single or repeated applications every 4-5 days to maintain the cat symptom-free. After symptoms abate, however, longer intervals may be acceptable between applications. When symptoms return, application may be repeated as necessary at the owner's discretion and based on the cat's response to the treatment.

This treatment is effective for a wide variety of feline skin disorders, relieving symptoms such as fuzzy, ragged, or otherwise abnormal fur, dandruff/skin flaking, and excessive shedding. Even on very excessive shedding, with loose fur that comes out in clumps on stroking, the treatment is very effective. The treatment is effective in relieving abnormal skin conditions and symptoms that ordinary feline care, such as routine coat brushing, does not resolve on its own.

Coconut oil, in addition to being unexpectedly very effective in treating feline skin disorders, has many inherent advantages over other treatments. It is natural and safe for human consumption and is routinely used in various recipe preparations and in cooking. In addition, it has been found to be safe for cats when consumed in small quantities, for example when a cat licks its fur for grooming after treatment. Cats may be poisoned by many chemicals that are usually considered safe by pet owners, because their livers are less effective at some forms of detoxification than those of many other animals, including humans and dogs. For example, minoxidil, a human hair-loss treatment, is highly toxic to cats and should never be administered. Many essential oils are also toxic to cats. Coconut oil, therefore, is very advantageous in comparison to other skin treatments that may need to be administered very carefully to avoid toxicity. It has a long shelf life, lasting a year or longer without degradation or rancidity when kept at room temperature (under 76 degrees Fahrenheit). It easily warms and melts in a user's hands for ease of application. Also, it has little odor, but the odor is pleasant and well-tolerated by cats.

One of its most unique advantages over other products is that, although it is called an “oil”, it does not have the consistency of what is usually considered an oil. It is not oily and not greasy. As previously described for its use, it melts easily and leaves no unpleasant residue on the user or the cat. Thus, fabrics, furniture, or carpets, will not be stained when in immediate contact with the cat or with the user's fingertips.

To improve the fragrance and/or appearance of the coconut oil, various other ingredients may be added. For aroma, various herbs can be added such as lavender, rosemary or thyme (which is known to be pleasing to some cats, as well as to humans). Other fragrant natural ingredients which are safe and edible can also be added, for example almond extract. To improve the appearance, various food colors or colored and/or textured ingredients can be mixed in. Glycerine, for example, can be mixed with the oil for a different texture and appearance. When adding additional ingredients, care must be taken to consider the effects on the product's shelf-life, as well as the dilution of the active ingredient and potential inhibition of absorption of the coconut oil into a cat's skin. A trace amount of coconut oil mixed into another preparation is not sufficient to achieve the beneficial results noted for coconut oil alone. In some embodiments, the proportion of coconut oil to an overall mixture may be 50% by weight or more.

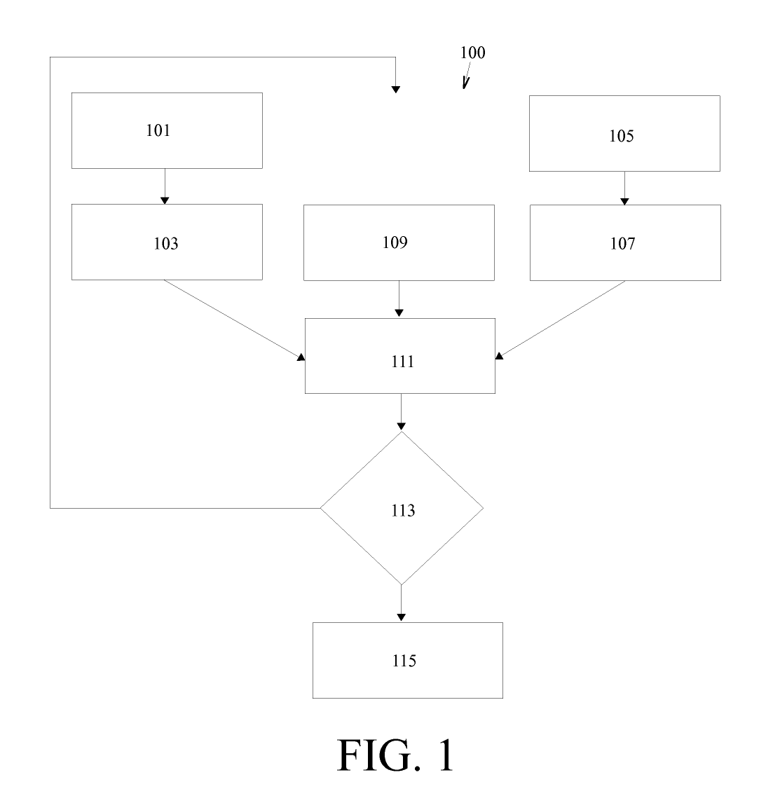

FIG. 1 is a flowchart illustrating a feline skin disorder treatment method 100, in an embodiment. Coconut oil is applied to a user's fingertips by running the tips of the middle three fingers of one hand across the surface of the coconut oil 109 until the fingertips are coated, applying coconut oil to the middle three fingertips of one of the user's hands 101 and then rubbing those fingertips against the fingertips on the user's other hand 103, or applying coconut oil to the middle three fingertips of one of the user's hands 105 and then rubbing the thumb of that hand against the other fingertips 107 to achieve a smooth and even coating. The user's body heat and friction from rubbing the coconut oil melts the coconut oil if it is in a solid form. The user then pushes his or her fingertips through a cat's fur and brushes them against the lie of the cat's fur across an area of abnormal skin on the cat 111 to apply the coconut oil thereto. The user may make three strokes against the skin of the cat with the fingers of both the user's hands. The user then checks to see if the coconut oil has been applied to the full area of abnormal skin 113. If not, the application of coconut oil to the user's fingertips and to the cat's skin may be repeated. If so, the user smooths the cat's fur down in the direction of growth 115. This method 100 may be repeated once daily until symptoms abate and then as needed or every 4-5 days.

The invention is not limited to the particular embodiments described above in detail. Those skilled in the art will recognize that other arrangements could be devised, for example, using various frequencies of application, both initial and ongoing, using various methods or implements for spreading the coconut oil on a cat's skin, and combining the coconut oil with various other ingredients, for example for improved scent, appearance, or texture. While the invention has been described with reference to specific illustrative embodiments, modifications and variations of the invention may be constructed without departing from the scope of the invention.

I claim:

1. A method of treating feline skin disorders, comprising:
applying coconut oil to the skin of a cat.

2. The method of claim 2, further comprising identifying an area of abnormal skin on the cat, wherein the applying coconut oil to the skin of a cat comprises applying the coconut oil to the area of abnormal skin.

3. The method of claim 1, wherein the applying coconut oil comprises applying coconut oil to a user's fingertips and then brushing the user's fingertips against the skin of the cat.

4. The method of claim 1, wherein the coconut oil is not mixed with any other ingredients.

5. The method of claim 1, wherein the coconut oil is mixed with one or more additional ingredients for aroma, appearance, or texture.

6. The method of claim 5, wherein the ingredients comprise an herb, food coloring, glycerine, or almond extract.

7. The method of claim 6, wherein the ingredients comprise lavender, rosemary or thyme.

8. The method of claim 5, wherein the coconut oil is 50% or more by weight of the mixture.

9. The method of claim 1, wherein the amount of coconut oil applied is less than ¼ of a teaspoon.

10. The method of claim 3, wherein the applying coconut oil to the user's fingertips comprises applying coconut oil to the fingertips of one of the user's hands and then rubbing those fingertips against the fingertips on the user's other hand, applying coconut oil to the fingertips of one of the user's hands and then rubbing the thumb of that hand against the other fingertips, or running the tip of the fingers of one hand across the surface of the coconut oil until the fingertips are coated.

11. The method of claim 1, further comprising melting the coconut oil before application.

12. The method of claim 3, wherein applying coconut oil to a user's fingertips comprises applying coconut oil to the fingertips of the middle three fingers of the user's hand.

13. The method of claim 3, wherein the brushing the user's fingertips against the skin of the cat comprises pushing the user's fingertips through the cat's fur until they contact the cat's skin.

14. The method of claim 3, wherein the brushing the user's fingertips against the skin of the cat comprises brushing against the lie of the cat's fur.

15. The method of claim 2, wherein applying the coconut oil to the area of abnormal skin comprises applying the coconut oil to the full area of abnormal skin.

16. The method of claim 3, further comprising smoothing the cat's fur down in the direction of growth.

17. The method of claim 3, wherein brushing the user's fingertips against the skin of the cat comprises making three strokes against the skin of the cat with the fingers of both the user's hands.

18. The method of claim 2, comprising repeating the application once daily until symptoms of skin disorder completely abate.

19. A feline skin disorder treatment composition, comprising:
coconut oil; and
one or more additional ingredients for aroma, appearance, or texture;
wherein the coconut oil is mixed with the one or more additional ingredients such that the coconut oil is 50% or more of the mixture by weight.

20. The composition of claim 19, further comprising instructions for applying the composition to a cat's skin to treat a feline skin disorder.
